SEQUENCE LISTING (20 sequences)

SEQ ID NO: 1 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 2 - protein, 347 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 3 - protein, 353 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 4 - protein, 353 aa, Homo sapiens
PEPELNKERPSLQIKLKIEDFELHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 5 - protein, 353 aa, Homo sapiens
PEPELNKERPSLQIKLKIEDFELHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLKKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 6 - protein, 353 aa, Homo sapiens
PEPELNKERPSLQIKLKIEDFELHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILEGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 7 - protein, 353 aa, Homo sapiens
PEPELNKERPSLQIKLKIEDFELHKMLGKGSFGKVFLAEFKKTNQFFAIKALKKDVVLEDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 8 - protein, 353 aa, Homo sapiens
PEPELNKERPSLQIKLKIEDFELHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLEFADRALINSMDQNMFRNFEFMNPGMERLISHHHHHH

SEQ ID NO: 9 - protein, 353 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNAFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 10 - protein, 353 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLEFADRALINSMDQNMFRNFEFMNPGMERLISHHHHHH

SEQ ID NO: 11 - protein, 353 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 12 - protein, 353 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLEFADRALINSMDQNMFRNFSFMNPGMERLISHHHHHH

SEQ ID NO: 13 - protein, 353 aa, Homo sapiens
PEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFEFMNPGMERLISHHHHHH

SEQ ID NO: 14 - protein, 391 aa, Homo sapiens
MSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 15 - protein, 371 aa, Homo sapiens
MGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 16 - protein, 347 aa, Homo sapiens
MERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 17 - protein, 347 aa, Homo sapiens
MERPSLQIKLKIEDFELHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 18 - protein, 341 aa, Homo sapiens
MIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 19 - protein, 338 aa, Homo sapiens
MKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 20 - protein, 338 aa, Homo sapiens
MKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNEFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLEFADRALINSMDQNMFRNFEFMNPGMERLISHHHHHH

(19) United States
(12) Patent Application Publication
(10) Pub. No.: US 2007/0072281 A1
(43) Pub. Date: Mar. 29, 2007

(54) Three-dimensional structure of the catalytic domain of protein kinase c theta, methods and uses thereof

(76) Inventors: Gabriele Rummel, Lorrach (DE); Wilhelm Stark, Therwil (CH); Andre Strauss, Langnau im Emmental (CH); Jurgen Wagner, Bottmingen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/547,273

(22) PCT Filed: Feb. 27, 2004
(86) PCT No.: PCT/EP04/01969

§ 371 Date: Sep. 11, 2006

Related U.S. Application Data
(60) Provisional application No. 60/450,780, filed on Feb. 28, 2003.

(51) Int. Cl. G06F 19/00; C12N 9/12

(52) U.S. Cl. 435/194; 702/19

(57) ABSTRACT

The present invention concerns protein kinase C (PKC) theta, in particular, the three-dimensional structure of the catalytic domain of PKC theta. Methods of expression, purification and crystallization of PKC theta catalytic domain are provided in preparation for crystallography. The atomic coordinates of a complex of PKC theta complexed with an inhibitor are also provided.

### FIELD OF THE INVENTION

The present invention relates to protein kinase C (PKC) theta, in particular, the three-dimensional structure of the catalytic domain of protein kinase C theta. The invention also relates to the crystalline forms of liganded or unliganded human protein kinase C theta catalytic domain. Further, the invention provides methods of expression and purification of the recombinant human PKC theta catalytic domain in preparation for crystallography, the crystallization of a complex of PKC theta with an inhibitor and its structural determination.

### BACKGROUND OF THE INVENTION

Currently, ten PKC isozymes are known, which are divided into three families based on their requirements for activation (Dekker et al., 1995; Newton, 2001). The classical cPKC alpha, beta1, beta2 and gamma require diacylglycerol (DAG), phosphatidylserine (PS) and calcium for activation. The novel nPKC delta, epsilon, eta and theta require DAG and PS, but are calcium insensitive, while the atypical aPKC iota, lambda and zeta require neither DAG nor calcium. All isoforms except PKC gamma and beta2 are expressed in T cells. The role of the PKC isoforms in T cell activation was reviewed recently (Wilkinson et al., 1998; Meller et al., 1998). The data suggest in particular that PKC theta plays a central role in T cell activation/proliferation.

PKC theta exhibits a restricted expression pattern with high protein levels found predominantly in hematopoietic cells and skeletal muscle (Meller et al., 1998). There is in vitro and in vivo evidence for the role of PKC theta in T cells. First, PKC theta is the only isoform which is selectively translocated to the T cell-antigen-presenting cell (APC) contact site immediately after cell-cell interaction (Monks et al., 1997 and 1998). Second, the induction of the IL-2 gene via activation of the NF-kappa B transcription factor requires fully functional PKC theta (Lin et al., 2000; Coudronniere et al., 2000; Dienz et al., 2000; Khoshnan et al., 2000). Finally, according to the phenotype of the PKC theta KO mice, PKC theta is essential for TCR-mediated activation of mature T cells (Sun et al., 2000). Several recent reviews describe in detail the role of PKC theta in T cells (Altmann et al., 2000; Arendt et al., 2002; Isakov et al., 2002).

All members of the PKC family of kinases have in common a conserved kinase core carboxyl terminal and a regulatory moiety. The regulatory moiety contains several domains. The C1 domain binds phosphatidylserine and diacylglycerol/phorbol esters. The C2 domains bind anionic lipids. PS is the pseudosubstrate domain, which prevents access of the substrate to the active site in the inactive conformation. Phosphorylation of threonine 538 in the activation loop by the upstream kinase PDK-1 is required to induce the active conformation of PKC theta. Subsequent to this first phosphorylation event, serines 676 and 695 are autophosphorylated.

Structural information is so far only available for the C1 domain (residues 231-280) of PKC delta and the C2 domains of PKC alpha (residues 155-293) PKC beta (residues 157-289) and PKC delta (residues 1-123) (Zhang et al. 1995; Pappa et al. 1998; Sutton et al. 1998 and Verdaguer et al. 1999, Williams & Mitchell 2002). No structure of a kinase domain of any member of the PKC family has been described so far.

### SUMMARY OF THE INVENTION

The present invention provides the three-dimensional structure of PKC-theta catalytic domain thereby enabling identification and design of ligands or low molecular weight molecules that specifically inhibit PKC theta.

The present invention relates to:
(i) a crystal of the PKC theta comprising the catalytic domain of PKC theta with or without a ligand or low molecular weight compound
(ii) methods of expressing PKC theta comprising the PKC theta catalytic domain
(iii) methods of purification of PKC theta comprising PKC theta catalytic domain
(iv) methods of making a crystal comprising the PKC theta catalytic domain
(v) methods of using said PKC crystal comprising the catalytic domain and its structural coordinates to identify and design ligands or low molecular weight molecules that inhibit PKC theta.

The three-dimensional structural information revealed from the crystal of the catalytic domain of PKC theta can be used for structure-based drug discovery, for screening, identifying and designing inhibitors of PKC theta and other PKC family members.

### DETAILED DESCRIPTION OF THE INVENTION

The full-length sequence of human PKC theta is given by SEQ ID No.1 (see: Genbank Accession number HUMPKCTH L07032.1, Swissprot Accession number Q04759, Baier et al. 1993 Chang et al. 1993/1994).

The present invention provides PKC theta catalytic domain in crystallized form. In particular, it provides a crystal comprising the catalytic domain of PKC theta and a ligand bound to PKC theta as a complex.

In one embodiment of the present invention, a crystal of the catalytic domain of PKC theta comprising a unit cell dimension of a=b=152.33±5 Ångstroms c=74.84±5 Ångstroms, α=90.0 degrees β=90.0 degrees γ=120 degrees is provided. Depending on the particular conditions for crystallization, the parameters characterising the unit cell may vary within a limited range, for example, a,b,c each vary by up to 5 Ångstroms.

The term “unit cell” according to the invention refers to the basic shape block. The entire volume of a crystal is constructed by regular assembly of such blocks. Each unit cell comprises a complete representation of the unit of pattern, the repetition of which builds up the crystal.

In another embodiment of the invention, a crystal of PKC theta comprising the catalytic domain of PKC theta in complex with a ligand is provided wherein said crystal has a three-dimensional structure characterized by the atomic structure coordinates of Table 1.

In a further embodiment of the invention, said catalytic domain of PKC theta comprises the sequence of SEQ ID. No. 2, a fragment or homologue thereof.

In another embodiment of the invention, said crystal comprises a mutant of the catalytic domain of PKC theta selected from the group of mutants SEQ ID No. 3 to SEQ ID No. 20, a fragment or homologue thereof.

In yet another embodiment of the invention, said catalytic domain of PKC theta comprises at least the ATP-binding site.

Further provided by this invention is a crystal comprising the catalytic domain of PKC theta (SEQ ID No. 2) bound to at least one ligand or low molecular weight compound.

The term “ligand” according to the invention, refers to a molecule or group of molecules that bind to one or more specific sites of PKC theta, preferably to the catalytic domain of PKC theta and most preferably to the ATP binding-site of said catalytic domain. Ligands according to the invention are preferably low molecular weight molecules.

The term “low molecular weight compound” according to the invention refers to preferably organic compounds generally having a molecular weight less than about 1000, more preferably less than about 600. Most preferably, said low molecular weight compounds or ligands inhibit PKC theta enzymatic activity.

In context of a PKC theta inhibitor, the terms “peptide” or “peptide derivative” are intended to embrace a “peptidomimetic” or “peptide analogue” which complement the three-dimensional structure of the binding site of PKC theta or can be designed with improved physical or chemical properties to bind with the three-dimensional binding site of the PKC theta catalytic domain as provided in the present invention.

The term “mutant” refers to differences within the wild-type sequence of PKC theta set forth in SEQ. ID No. 1 by deletion, insertion or preferably replacement of one or more selected amino acids.

According to the present invention, the term “mutant” also refers to a polypeptide, whose amino acid sequence differs from the wild-type sequence given in SEQ ID No.2 by deletion, insertion or preferably replacement of one or more selected amino acids, said mutant being at least 50% homologous to SEQ ID No. 2, more preferably at least 80% homologous to SEQ ID No. 2 most preferably at least 90% homologous to SEQ ID No. 2. For example, a PKC theta mutant of the catalytic domain of the present invention is preferably a mutant from the group of SEQ ID. No. 3 to SEQ.ID.No. 20.

A “fragment” of PKC theta catalytic domain according to the invention comprises more than 50% of the PKC theta catalytic domain according to SEQ ID No. 2, more preferably at least 80% of the PKC theta catalytic domain according to SEQ ID No. 2, most preferably at least 90% of the PKC theta catalytic domain according to SEQ ID No. 2.

An “N-terminal extension” of the PKC theta catalytic domain according to the invention comprises the addition of 1 to 359 amino acids from the N-terminus of the full-length PKC theta (SEQ ID No. 1) to the N-terminus of the catalytic domain of PKC theta.

In one embodiment of the invention, a PKC theta mutant of the catalytic domain may be crystallizable with or without at least one ligand.

In another embodiment of the invention, a PKC theta fragment of the catalytic domain may be crystallizable with or without at least one ligand.

In yet another embodiment of the invention, a method is provided wherein the catalytic domain of PKC theta (SEQ ID No. 2), a mutant (SEQ ID No. 3-SEQ ID No. 20), fragment or homologue of the catalytic domain of PKC theta is bound to at least one ligand at any step prior to crystallization.

According to the present invention, PKC theta crystals are stable if kept under suitable conditions. For example, the crystals are stable in there mother liquor at 4° C. for at least 3-4 weeks. Preferable storage is frozen in liquid nitrogen.

In the present invention, PKC theta and preferentially the catalytic domain of PKC theta, a fragment or homologue thereof is advantageously obtained by expressing PKC theta protein in a recombinant baculo-virus infected insect cell culture in the presence of an inhibitor of PKC theta. Alternatively, the PKC theta domain may also be obtained by expression of the PKC theta full-length protein and subsequent proteolytic cleavage of the PKC theta catalytic domain or by co-expression of the PKC theta catalytic domain with a pseudosubstrate.

In one embodiment of the invention, a method for making a crystal of a PKC theta is provided comprising the following steps:
(i) purification of the full-length PKC theta (SEQ ID No.1)
(ii) expression of the full-length PKC theta (SEQ ID No. 1) or catalytic domain of PKC theta (SEQ ID No. 2) in a suitable host cell
(iii) purification of the desired PKC theta domain.

In a preferred embodiment of the invention, said method for making a crystal involves the catalytic domain of PKC theta (SEQ ID No.2), a mutant (SEQ ID No. 3-SEQ ID No. 20), a fragment or homologue of the catalytic domain of PKC theta.

In a preferred embodiment of the invention, the method of making a crystal involves expression of the desired domain of PKC theta in the presence of a PKC theta inhibitor.
A further preferred embodiment of the invention involves the method of making a crystal wherein the catalytic domain of PKC theta is phosphorylated at sites Serine 676 or Serine 695 or at both sites.

According to the invention, PKC theta may be prepared by isolation from natural sources, e.g. cultured human cells or preferably by recombinant heterologous expression. Expression of recombinant PKC theta is achievable in eukaryotic or prokaryotic systems. For example, recombinant human PKC theta may be expressed in insect cells, such as Sf9 cells, using a suitable recombinant baculovirus system or in bacteria.

The kinase may be expressed as a fusion protein, e.g. a glutathione-S-transferase (GST) or histidine-tagged fusion protein or as an untagged protein. If desired, the fusion partner is removed before crystallization. The heterologously produced PKC theta to be used for crystallization is potentially biologically active.

Methods for the preparation of PKC theta mutants are commonly known in the art. For example, PKC theta mutants may be prepared by expression of PKC theta DNA previously modified in its coding region by oligo-nucleotide directed mutagenesis.

In the present invention, purified PKC theta is preferably at least 90% homogeneous. Protein homogeneity is determinable according to analytical methods well-known in the art, e.g. sequence analysis, electrophoresis, spectroscopic or chromatographic techniques. The purified protein is potentially enzymatically active. Appropriate assays for determining PKC theta activity towards a suitable substrate, e.g. a natural substrate or a synthetic substrate which are known in the art.

In one embodiment of the invention, at any step prior to crystallization PKC theta may be complexed with a low molecular weight compound or ligand which is capable of suitably binding to the PKC theta catalytic domain site. Preferred is a compound inhibiting PKC theta activity. Kinase inhibition is determinable employing assays known in the art. Suitable inhibitors include ATP-competitive kinase inhibitors which act on the catalytic domain to inhibit PKC theta activity.

Various methods of crystallization can be used in the claimed invention including vapor diffusion, dialysis or batch crystallization. In vapor diffusion crystallization, a small volume (i.e., a few microliters) of protein solution is mixed with a solution containing a precipitant. This mixed volume is suspended over a well containing a small amount, i.e. about 1 ml, of precipitant. Vapor diffusion between the drop and the well will result in crystal formation in the drop.

The dialysis method of crystallization utilizes a semipermeable size-exclusion membrane that retains the protein but allows small molecules (i.e. buffers and precipitants) to diffuse in and out. In dialysis, rather than concentrating the protein and the precipitant by evaporation, the precipitant is allowed to slowly diffuse through the membrane and reduce the solubility of the protein while keeping the protein concentration fixed.

The batch method generally involves the slow addition of a precipitant to an aqueous solution of protein until the solution just becomes turbid, at this point the container can be sealed and left undisturbed for a period of time until crystallization occurs. In the batch technique the precipitant and the target molecule solution are simply mixed. Supersaturation is achieved directly rather than by diffusion. Often the batch technique is performed under oil. The oil prevents evaporation and extremely small drops can be used. For this, the term “microbatch” is used. A modification of this technique is not to use paraffin oil (which prevents evaporation completely) but rather use silicone oil or a mixture of silicone and paraffin oils so that a slow evaporation is possible.

The claimed invention can encompass any and all methods of crystallization. One skilled in the art can choose any of such methods and vary the parameters such that the chosen method results in the desired crystals.

One preferred method of crystallization of PKC theta involves mixing a PKC theta solution with a “reservoir buffer”, with a lower concentration of the precipitating agent necessary for crystal formation. For crystal formation, the concentration of the precipitating agent has to be increased, e.g. by addition of precipitating agent, for example by titration, or by allowing the concentration of precipitating agent to balance by diffusion between the crystallization buffer and a reservoir buffer. Under suitable conditions such diffusion of water or volatile precipitating agent occurs along the gradient of precipitating agent, e.g. between the reservoir buffer having a higher concentration of precipitating agent and the crystallization buffer having a lower concentration of precipitating agent. Diffusion may be achieved e.g. by vapour diffusion techniques allowing diffusion of water in the common gas phase. Known techniques are e.g. vapour diffusion methods, such as the “hanging drop” or the “sitting drop” method. In the vapour diffusion method a drop of crystallization buffer containing the protein is hanging above or sitting beside a much larger pool of reservoir buffer. Alternatively, the balancing of the precipitating agent can be achieved through a semipermeable membrane (dialysis method) that separates the crystallization buffer from the reservoir buffer and prevents dilution of the protein into the reservoir buffer.

Formation of PKC theta catalytic domain crystals can be achieved under various conditions which are essentially determined by the following parameters: pH, presence of salts and additives, precipitating agent, protein concentration and temperature. The pH may range, for example, from about 4.0 to 9.0.

The present invention also relates to a computer readable medium having stored a model of the PKC theta catalytic domain crystal structure. In a preferred embodiment, said model is built from all or part of the X-ray diffraction data. The atomic coordinates are shown in Table 1.

The present invention provides the structure coordinates of human PKC theta catalytic domain. The term “structure coordinates” or “atomic coordinates” refers to mathematical coordinates derived from the mathematical equations (fourier transformation) related to the diffraction pattern obtained on a monochromatic beam of X-rays by the atoms (scattering centers) of a crystal comprising a PKC theta catalytic domain. The diffraction data are used to calculate an electron density map of the repeating unit of the crystal. The electron density maps are used to establish the positions of the individual atoms within the unit cell of the crystal.

Structural coordinates of a crystalline composition of this invention may be stored in a machine-readable form on a machine-readable storage medium, e.g. a computer hard drive, diskette, DAT tape, etc., for display as a three-dimensional shape or for other uses involving computer-assisted manipulation of, or computation based on, the structural coordinates or the three-dimensional structures they define. For example, data defining the three dimensional structure of a protein of the PKC family, or portions or structurally similar homologues of such proteins, may be stored in a machine-readable storage medium, and may be displayed as a graphical three-dimensional representation of the protein structure, typically using a computer capable of reading the data from said storage medium and programmed with instructions for creating the representation from such data.

In one embodiment of the invention, a computer readable medium is provided comprising data storage material encoded with computer readable data wherein said data comprises the atomic coordinates of Table 1 comprising the catalytic domain of PKC theta.

In another embodiment of the invention, a method is provided for determining the three-dimensional structure of the catalytic domain of PKC theta comprising:
(i) crystallization of PKC theta comprising the catalytic domain of PKC theta (SEQ ID No.2), a mutant (SEQ ID No. 3-SEQ ID No. 20), fragment or homologue thereof
(ii) collecting x-ray diffraction data in the form of indexed intensities for said crystal and generating atomic coordinates
(iii) utilizing the atomic coordinates of Table 1 in whole or in part to determine the three-dimensional structure of the catalytic domain of PKC theta, fragment, or homologue thereof.

In yet another embodiment of the invention, a method is provided for determining the three-dimensional structure of a complex comprising the catalytic domain of PKC theta (SEQ ID No.2), a mutant (SEQ ID No. 3-SEQ ID No. 20) fragment or homologue thereof bound to at least one ligand comprising the steps of:
(i) obtaining x-ray diffraction data for a crystal of the complex
(ii) utilizing the atomic coordinates of Table 1 in whole or in part to determine the three-dimensional structure of the complex.

According to the present invention, a three-dimensional PKC theta model is obtainable from a PKC theta crystal comprising the catalytic domain of PKC theta, mutant, fragment or homologue thereof. Such a model can be built or refined from all or part of the PKC theta structure data of the present invention using the x-ray diffraction coordinates, particularly the atomic structure coordinates of Table 1.

The knowledge obtained from the three-dimensional model of the catalytic binding site of PKC theta can be used in various ways. For example, it can be used to identify chemical entities, for example, small organic and bioorganic molecules such as peptidomimetics and synthetic organic molecules that bind to PKC theta and preferably block or prevent a PKC theta mediated or associated process or event, or that act as PKC theta agonists. Using the three-dimensional structure of the PKC theta catalytic domain, the skilled artisan constructs a model of the PKC theta. For example, every atom can be depicted as a sphere of the appropriate van der Waals radius, and a detailed surface map of the PKC theta catalytic domain can be constructed.

Chemical entities that have a surface that mimics the accessible surface of the catalytic binding site of PKC theta can be constructed by those skilled in the art. By way of example, the skilled artisan can screen three-dimensional structural databases of compounds to identify those compounds that position appropriate functional groups in similar three dimensional structural arrangement, then build combinatorial chemistry libraries around such chemical entities to identify those with high affinity to the catalytic binding site of PKC theta.

In one embodiment of the invention, a method is provided for identifying a ligand or low molecular weight compound that binds to the catalytic domain of PKC theta comprising:
(i) using the three-dimensional structure of the catalytic domain derived in whole or in part from the set of atomic coordinates in Table 1
(ii) selecting a ligand or low molecular weigh compound that binds to the catalytic domain of PKC theta

In another embodiment of the invention, a method is provided for identifying a ligand or low molecular weight compound that binds to the catalytic domain of PKC theta wherein the catalytic domain of PKC theta comprises at least the ATP binding site of said domain.

In yet another embodiment of the invention, a method is provided for identifying ligands which inhibit the activity of PKC theta.

Ligands or small molecular compounds can be identified from screening compound databases or libraries and using a computational means to form a fitting operation to a binding site on the catalytic domain of PKC theta. The three dimensional structure of the catalytic domain of PKC theta as provided in the present invention in whole or in part by the structural coordinates of Table 1, can be used together with various docking programs.

The potential inhibitory or binding effect of a chemical entity on PKC theta may be analyzed prior to its actual synthesis and testing by the use of computer-modeling techniques. If the theoretical structure of the given chemical entity suggests insufficient interaction and association between it and PKC theta, the need for synthesis and testing of the chemical entity is obviated. However, if computer modeling indicates a strong interaction, the molecule may then be synthesized and tested for its ability to bind to PKC theta. Thus, expensive and time-consuming synthesis of inoperative compounds may be avoided.

An inhibitory or other binding compound of PKC theta may be computationally evaluated and designed by means of a series of steps in which chemical entities or fragments are screened and selected for their ability to associate with the individual binding sites of PKC theta. Thus, one skilled in the art may use one of several methods to screen chemical entities or fragments for their ability to associate with PKC theta. This process may begin by visual inspection of, for example, the binding site on a computer screen based on the structural coordinates of Table 1 in whole or in part. Selected fragments or chemical entities may then be positioned in a variety of orientations, or “docked,” within the catalytic binding site of PKC theta. Docking may be accomplished using software such as Quanta and SYBYL, followed by energy minimization and molecular dynamics with standard molecular mechanics force fields, such as CHARMM and AMBER. Specialized computer programs may be of use for selecting interesting fragments or chemical entities. These programs include, for example, GRID, available from Oxford University, Oxford, UK; 5 MCSS or CATALYST, available from Molecular Simulations, Burlington, Mass.; AUTODOCK, available from Scripps Research Institute, La Jolla, Calif.; DOCK, available from University of California, San Francisco, Calif., and XSITE, available from University College of London, UK.

Using molecular replacement to exploit a set of coordinates such as those of Table 1 of the invention, the structure of a crystalline PKC theta catalytic domain or portion thereof can for example, be bound to one or more ligands or low molecular weight compounds to form a complex.

The term “molecular replacement” refers to a method that involves generating a preliminary structural model of a crystal whose structural coordinates are unknown, by orienting and positioning a molecule whose structural coordinates are known, e.g., the PKC theta catalytic domain coordinates within the unit cell of the unknown crystal, so as to best account for the observed diffraction pattern of the unknown crystal. Phases can then be calculated from this model, and combined with the observed amplitudes to give an approximated Fourier synthesis of the structure whose coordinates are unknown. This in turn can be subject to any of the several forms of refinement to provide a final accurate structure. Using the structural coordinates provided by this invention, molecular replacement may be used to determine the structural coordinates of a crystalline co complex, unknown ligand, mutant, or homolog, or of a different crystalline form of PKC theta. Additionally, the claimed crystal and its coordinates may be used to determine the structural coordinates of a chemical entity that associates with PKC theta.

“Homology modeling” according to the invention involves constructing a model of an unknown structure using structural coordinates of one or more related proteins, protein domains and/or one subdomains such as the catalytic domain of PKC theta. Homology modeling may be conducted by fitting common or homologous portions of the protein or peptide whose three dimensional structure is to be solved to the three dimensional structure of homologous structural elements. Homology modeling can include rebuilding part or all of a three dimensional structure by replacement of amino acids or other components by those of the related structure to be solved.

Molecular replacement according to the present invention, uses a molecule having a known structure. The three-dimensional structure of the catalytic domain of PKC theta provided in whole or in part in Table 1 in a machine-readable form on a data-carrier can be used as a starting point to model the structure of an unknown crystalline sample. This technique is based on the principle that two molecules which have similar structures, orientations and positions in the unit cell diffract similarly. Molecular replacement involves positioning the known structure in the unit cell in the same location and orientation as the unknown structure. Once positioned, the atoms of the known structure in the unit cell are used to calculate the structure factors that would result from a hypothetical diffraction experiment. This involves rotating the known structure in the six dimensions (three angular and three spatial dimensions) until alignment of the known structure with the experimental data is achieved. This approximate structure can be fine-tuned to yield a more accurate and often higher resolution structure using various refinement techniques. For instance, the resultant model for the structure defined by the experimental data may be subjected to rigid body refinement in which the model is subjected to limited additional rotation in the six dimensions yielding positioning shifts of under about 5%. The refined model may then be further refined using other known refinement methods. The present invention also enables homologues and mutants of PKC theta catalytic domain and the solving of their crystal structure. Based on the three-dimensional structure of PKC theta catalytic domain as provided in the present invention and using the atomic coordinates of Table 1 in whole or in part, the effects of site-specific mutations can be predicted. More specifically, the structural information provided herein permits the identification of desirable sites for amino acid modification, particularly amino acid mutation resulting in substitutional, insertional or deletional variants. Such variants may be designed to have special properties, particularly properties distinct from wild-type PKC theta catalytic domain, such as altered catalytic activity. Substitutions, deletions and insertions may be combined to arrive at a desired variant. Such variants can be prepared by methods well-known in the art, e.g. starting from wild-type PKC theta catalytic domain, or by de novo synthesis.

PKC theta catalytic domain may also crystallize in a form different from the one disclosed herein. The structural information provided, for example, in SEQ ID No. 2 and Table 1 in whole or in part, is also useful for solving the structure of other crystal forms. Furthermore, it may serve to solve the structure of a PKC theta catalytic domain mutant, a PKC theta catalytic domain co-complex or a sufficiently homologous protein.

The PKC theta catalytic domain structural information provided herein is useful for the design of ligands or small molecule compounds which are capable of selectively interacting with PKC theta catalytic domain and thereby specifically modulating the biological activity of PKC theta. Furthermore, this information can be used to design and prepare PKC theta mutants, e.g. mutants with altered catalytic activity, model the three-dimensional structure and solve the crystal structure of proteins, such as PKC theta catalytic domain homologues, PKC theta catalytic domain mutants or PKC theta catalytic domain co-complexes, involving e.g. molecular replacement.

The present invention provides a method for designing a ligand or low molecular weight compound capable of binding with PKC theta catalytic domain, said method comprising:
(i) using the atomic coordinates of Table 1 in whole or in part to determine the three-dimensional structure of the PKC theta catalytic domain
(ii) probing said three-dimensional structural of the PKC theta catalytic domain with candidate ligands or low molecular weight compounds to determine which bind to the catalytic domain of PKC theta
(iii) selecting those ligands or low molecular weight compounds which bind to the catalytic domain of PKC theta
(iv) modifying those ligands or low molecular weight compounds which bind to maximize physical properties such as solubility, affinity, specificity or potency.

Preferred is a method for designing a PKC theta inhibitor which interacts at the catalytic binding site. The present invention also relates to the chemical entity or ligand identified by such method. The present invention may also be used to design ligands or low molecular weight compounds which bind to another PKC family member using the atomic coordinates of Table 1 in whole or in part to determine the three-dimensional structure of a PKC family member's catalytic domain.

One approach enabled by this invention is the use of the structural coordinates of PKC theta catalytic domain to design chemical entities that bind to or associate with PKC theta and alter the physical properties of the chemical entities in different ways. Thus, properties such as, for example, solubility, affinity, specificity, potency, on/off rates, or other binding characteristics may all be altered and/or maximized. One may design desired chemical entities by probing an PKC theta crystal comprising the catalytic domain with a library of different entities to determine optimal sites for interaction between candidate chemical entities and PKC theta. For example, high-resolution x-ray diffraction data collected from crystals saturated with solutes allows the determination of where each type of solute molecule adheres. Small molecules that bind tightly to those sites can then be designed and synthesized and tested for the desired activity. The invention also contemplates computational screening of small-molecule databases or designing of chemical entities that can bind in whole or in part to PKC theta catalytic domain. They may also be used to solve the crystal structure of mutants, co-complexes, or the crystalline form of any other molecule homologous to, or capable of associating with, at least a portion of PKC theta. One method that may be employed for this purpose is molecular replacement. An unknown crystal structure, which may be any unknown structure, such as, for example, another crystal form of PKC theta catalytic domain, an PKC theta catalytic domain mutant or peptide, or a co-complex with PKC theta, or any other unknown crystal of a chemical entity that associates with PKC theta that is of interest, may be determined using the whole of part of the structural coordinates set forth in Table 1. This method provides an accurate structural form for the unknown crystal far more quickly and efficiently than attempting to determine such information without the invention herein.

In one preferred embodiment of the invention, candidate ligands are screened in silico. The information obtained can thus be used to obtain maximally effective inhibitors or agonists of PKC theta.

In another preferred embodiment of the invention, a method is provided to design ligands which inhibit the activity of PKC theta.

The design of chemical entities that inhibit or agonize PKC theta generally involves consideration of at least two factors. First, the chemical entity must be capable of physically or structurally associating with PKC theta, preferably at the catalytic site of PKC theta. The association may be any physical, structural, or chemical association, such as, for example, covalent or non-covalent binding, or van der Waals, hydrophobic, or electrostatic interactions. Second, the chemical entity must be able to assume a conformation that allows it to associate with PKC theta, preferentially at the catalytic site of PKC theta. Although not all portions of the chemical entity will necessarily participate in the association with PKC theta, those non-participating portions may still influence the overall conformation of the molecule. This in turn may have a significant impact on the desirability of the chemical entity.

Such conformational requirements include the overall three-dimensional structure and orientation of the chemical entity in relation to all or a portion of the binding site.

Once a compound has been designed or selected by the above methods, the efficiency with which that compound may bind to PKC theta may be tested and modified for the maximum desired characteristic(s) using computational or experimental evaluation. Various parameters can be maximized depending on the desired result. These include, but are not limited to, specificity, affinity, on/off rates, hydrophobicity, solubility, and other characteristics readily identifiable by the skilled artisan.

The present invention also relates to identification of compounds which modulate PKC theta. Preferred are compounds which inhibit PKC theta activity. In particular, said compounds are useful in preventing or treating disorders mediated by T-lymphocytes and/or PKC, for example, acute or chronic rejection or organ or tissue allografts or xenografts, atherosclerosis, vascular occlusion due to vascular injury such as angioplasty, restenosis, hypertension, heart failure, chronic obstructive pulmonary disease, CNS disease such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock or traumatic shock. Further, said compounds can be useful in preventing or treating T-cell mediated acute or chronic inflammatory diseases or disorders or autoimmune diseases, for example, rheumatoid arthritis, osteoarthritis, systemic lupus erythematosus, Hashimoto's thyroidis, multiple sclerosis, myasthenia gravis, diabetes type I or II and disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis, and further eczematous dermatitis, seberrhoeic dermatitis), inflammatory eye diseases, e.g. Sjoegren's syndrome, keratoconjunctivitis, or uveitis, inflammatory bowel disease, Crohn's disease or ulcerative colitis. An example of a such a PKC modulating compound can be found in WO 0238561.

For the above uses, the required dosage will depend on the mode of administration, the particular condition to be treated and the desired effect. In general, satisfactory results are indicated to be obtained systematically at daily dosages from about 0.1 to about 100 mg/kg body weight. An indicated daily dosage in a larger mammal (e.g. human) is in the range from about 0.5 mg to about 2000 mg, conveniently administered, for example, in divided doses up to four times a day or in retard form. The compounds may be administered by any conventional route, in particular enterally e.g. orally, e.g. in the form or tablets or capsules, or parenterally, e.g. in the form of injectable solutions or suspensions, topically, e.g. in the form of lotions, gels, ointments or creams, or in a nasal or a suppository form. Pharmaceutical compositions comprising said compound in free form or in a pharmaceutically acceptable salt form in association with at least one pharmaceutically acceptable carrier or diluent may be manufactured in conventional manner by mixing with a pharmaceutically acceptable carrier or diluent. Unit dosage forms for oral administration contain, for example, from about 0.1 mg to about 500 mg of active substance. Topical administration is e.g. to the skin. A further form of topical administration is to the eye. Compounds of the formula I may be administered in free form or in a pharmaceutically acceptable salt form. Such salts may be prepared in a conventional manner and exhibit the same order of activity as the free compounds.

The present invention enables the use of molecular design techniques, particularly the rational drug design approach, to prepare new or improved chemical entities and compounds, including PKC theta inhibitors, capable of irreversibly or reversibly, modulating PKC theta activity. Improved entities or compounds means that these entities or compounds are superior to the “original” or parent compound they are derived from with regard to a property relevant to therapeutic use including suitability for in vivo administration, e.g. cellular uptake, solubility, stability against (enzymatic) degradation, binding affinity or specificity, and the like. For example, on the basis on the information provided herein it is possible to specially design PKC theta inhibitors which covalently, or preferably non-covalently, bind to PKC theta. Such inhibitors may act in a competitive or uncompetitive manner, bind at or close to the active site of PKC theta or act allosterically.

In the design of PKC theta modulators the following aspects should be considered: (i) if the candidate compound is capable of physically and structurally associating with PKC theta catalytic domain, and/or (ii) if the compound is capable of assuming a conformation allowing it to associate with PKC theta catalytic domain. Advantageously, computer modelling techniques are used in the process of assessing these abilities for the modulator as a whole, or a fragment thereof—in order to minimize efforts in the synthesis or testing of insuccessful candidate compounds. Specialized computer software is well-known in the art. Another design approach is to probe a PKC theta catalytic domain crystal with a variety of different chemical entities to determine optimal sites for interaction between candidate PKC theta inhibitors and the target enzyme. Yet another possibility which arises from the present invention is to screen computationally small molecule data bases for chemical entities or compounds that are capable of binding, in whole or in part, to PKC theta catalytic domain. The quality of fit to the binding site may be judged e.g. by shape complementarity or by estimated interaction energy. Knowledge of the three-dimensional arrangement of the modifications can be then utilized for the design of new PKC theta ligands or low molecular weight compounds such as selective inhibitors.

Chemical entities that are capable of associating with the PKC family member may inhibit its interaction with naturally occurring ligands of the protein and may inhibit biological functions mediated by such interaction. In the case of PKC theta, such biological functions include activation of T cells during an immune response. Such chemical entities are potential drug candidates.

Compounds of the structures selected or designed by any of the foregoing means may be tested for their ability to bind to a PKC family protein, inhibit the binding of a PKC family protein to a natural or non-natural ligand therefore, and/or inhibit a biological function mediated by a PKC family member.

The following examples serve to illustrate embodiments of the present invention but should not be construed as a limitation thereof. Compounds identified by any of the methods described herein are also encompassed by this invention.

### EXAMPLES

Standard protocols in molecular biology (Ausubel et al. 1997) are applied, if not otherwise stated. The manufacturer's recommendations are followed when using commercially available kits. All primers are from Microsynth GmbH, Balgach, Switzerland. PCR products are purified with the PCR purification kit (Qiagen). After restriction enzyme digestion, fragments are run and extracted from agarose gels using the QIAquick gel extraction kit (Qiagen). Ligations are performed overnight at 16° C. with T4 ligase (BRL) and aliquots of the ligation mixtures are transformed into E. coli strain DH5α (Gibco) according to standard protocols for CaCl₂ competent cells.

### Example 1

### Generation of PKC Theta Mutants for Expression in E. coli

Several mutants, including a constitutively active mutant (T538E) are created in order to enhance the solubility of the expressed recombinant protein in E. coli. The first plasmid used for these experiments codes for the catalytic domain of PKC theta with some flanking amino acids, extending from proline 360 to serine 706 and followed by a C-terminal 6-histidine tag. The catalytic domain and the 6-histidine tag are excised from this plasmid with restriction endonucleases Nde1 and Hind3 and cloned into the corresponding sites of plasmid pET26b (Novagen) resulting in construct C#345. This construct is expressed in E. coli initiated at an internal ribosome binding site in the PKC theta. Therefore, this plasmid is submitted to a first mutagenesis with the primers RS463 and RS464 in order to destroy the Shine-Dalgarno consensus before M385 and, simultaneously, to introduce mutation I381E [C#368]. The Shine-Dalgarno consensus sequence is a ribosomal recognition sequence specific to prokaryotes (E-coli). The mutation I381E is introduced to enhance the solubility of the protein. The mutagenesis is performed with the QuikChange® Site Specific Mutagenesis kit (Stratagene) using 50 ng template DNA and 250 ng primers (HPLC purified). All successive mutagenesis is performed following the same protocol and using plasmid C#368 as template. All constructs have the mutation I381E and a C-terminal 6-His tag.

In total, 17 constructs are created and expressed in E. coli. From these plasmids, three mutants (I381E/M418E, I381E/L552E and I381E/V611K) and the constitutively active mutant (I381E/T538E) are selected for further expression with the baculovirus/insect cell system.

The effect of the regulatory domain of PKC theta (E135-P168) on co-expression of the catalytic domain is evaluated. The construct expressing this domain is created by PCR amplification using the full length PKC theta template and primers RS409 and RS410. The product is then digested with restriction endonuclease BamH1 and Not1 and cloned into the corresponding sites of pGEX-6P (Pharmacia) thus bringing the target gene in fusion with a GST tag (C#343).

The following is a summary of the constructs:

| | Introduced | Resulting | Name of protein | Sequence |
|---|---|---|---|---|
| Template | mutation | construct | mutant | ID No |
| C#368 | T538E | C#396 | M1c | 4 |
| C#368 | V611K | C#392 | M1d | 5 |
| C#368 | L552E | C#381 | M1e | 6 |
| C#368 | M418E | C#380 | M1f | 7 |

Many of the E. coli mutants are based on an in silico analysis of the catalytic domain and of the residues which upon mutations might increase the probability to obtain soluble protein upon bacterial expression. A significant increase of soluble expression is not observed.

### Example 2

### Generation of PKC Theta Mutants for Expression in the Baculovirus System

Bacterial constructs encoding the four selected double mutants (I381E/M418E, I381E/T538E, I381E/L552E and I381E/V611K) are modified for expression in the baculovirus/insect cell system. Their coding sequences are amplified by PCR with primer RS497 which adds a “Kozak” sequence immediately before the ATG initiation codon and RS68. The resulting products are then digested with restriction endonuclease Xho1 and Not1 and cloned under a polyhedrin promotor into the corresponding sites of pBacPAK8 (Clontech). In a similar manner, the regulatory domain (C#343) is amplified by PCR with primer RS501, adding a “Kozak” sequence before the ATG initiation codon of GST, and RS417. The resulting product is digested with restriction endonuclease Xho1 and Not1 and cloned into the corresponding sites of pBacPAK8.

In addition, the wild type catalytic domain of PKC theta is modified for untagged expression in baculovirus/insect cells with primer RS497 and primer RS421 which removes the 6-His tag at C-terminus. All plasmids can be sequenced using the ABI PRISM®R377 DNA sequencer and the ABI PRISM® BigDye™ Terminator Cycle Sequencing kit (Perkin Elmer Applied Biosystems).

The following is an overview of the constructs:

| | Domain/ | Baculo | Name of | Sequence |
|---|---|---|---|---|
| Template | Encoded mutations | construct | protein mutant | ID No |
| C#345 | 360-706 | C#436 | M1b | 2 |
| C#396 | 360-706-6His | C#435 | M1c | 4 |
| | I381E/T538E | | | |
| C#392 | 360-706-6His | C#434 | M1d | 5 |
| | I381E/V611K | | | |
| C#381 | 360-706-6His | C#410 | M1e | 6 |
| | I381E/L552E | | | |
| C#380 | 360-706-6His | C#433 | M1f | 7 |
| | I381E/M418E | | | |

In baculovirus infected insect cells, a suitable N-termini for the catalytic domain is identified by shortening systematically the linker region preceeding the domain and estimating the effect obtained by phosphorylation of the known Thr538 and Ser676 and Ser695 residues on solubility and stability of the catalytic domain upon expression in the insect cells. To achieve this, Thr and Ser residues are replaced with Glu which should partially mimick the phosphates in different combinations. In order to facilitate purification, most of constructs are produced with a C-terminal 6His tag. In most cases where His tags are introduced, they are not visible in the structure since they are usually disordered. Mutations such as I381E may be introduced in order to decrease the hydrophobicity at the surface of the kinase domain. I381E is located in the N-terminal domain and is solvent accessible. This residue should not disturb the conformation of the molecule.

### Example 3

### Plasmids for Baculovirus Infected Insect Cells Encoding Catalytic Domains

The plasmids encoding the wild type PKC domains that start at different positions in front of the catalytic domain are created by PCR fragment insertion mutagenesis. The template DNA for the PCR amplifications represents the coding sequence of the full wild type coding sequence of PKC theta. Oligonucleotides covering the newly wanted N-terminus of the PKC theta domain and the complementary oligonucleotides covering the C-terminal part of the kinase are elongated at their 5′ end with 33 or 42 nucleotides. Covering the junctions of the site in pXI338 between which the PKC theta domain is inserted in the final recombinant plasmid. The different mutants of the PKC theta kinase are all obtainable either by classical mutagenesis using the Stratagene QuikChange™ site specific mutagenesis kit or by PCR fragment insertion mutagenesis by using the pXI342c plasmid as template. All the inserts in the plasmids are checked for correctness by DNA sequencing.

The following is an overview of the plasmids:

Plasmids 1

| | Catalytic domain (amino | Amino acid at N- | | |
|---|---|---|---|---|
| | acid positions of wild | terminus of catalytic | Name of protein | Sequence |
| Plasmid | type sequence) | domain | mutant | ID No |
| pXI342 a | 323-706 | MSIKN | M5a | 14 |
| pXI342 b | 343-706 | MGISW | M6a | 15 |
| pXI342 d | 367-706 | MERPS | M7a | 16 |
| pXI342 e | 373-706 | MIKLKI | M8a | 18 |
| pXI342 f | 376-706 | MKIEDF | M9a | 19 |

Plasmids 2

| | Catalytic domain (amino | | Name of | |
|---|---|---|---|---|
| | acid positions of wild type | | protein | Sequence |
| Plasmid | sequence) | mutations | mutant | ID No |
| pXI342 g | 366-706 (MERPS . . . (His)₆) | I381E, T538E | M7b | 17 |
| pXI342 h | 375-706 (MKIEF . . . (His)₆) | T538E, S676E, S695E | M9b | 20 |
| pXI342 i | 360-706 (MPEPEL . . . (His)₆) | T538E, S676E, S695E | M1g | 8 |
| pXI342 k | 360-706 (MPEPEL . . . (His)₆) | T538A | M1h | 9 |
| pXI342 l | 360-706 (MPEPEL . . . (His)₆) | S676E, S695E | M1i | 10 |
| pXI342 m | 360-706 (MPEPEL . . . (His)₆) | T538E | M1k | 11 |
| pXI342 n | 360-706 (MPEPEL . . . (His)₆) | T538E, S676E | M1l | 12 |
| pXI342 o | 360-706 (MPEPEL . . . (His)₆) | T538E, S695E | M1m | 13 |

### Example 4

### Expression of PKC-Theta in Bacolovirus and Insect Cells

Methods for preparation of Baculovirus and insect cell cultures are known in the art (O'Reilly et al., 1994). The majority of recombinant Baculovirus is produced by transfection of Sf9 cells using the BacPAK6 transfection system from CLONTECH and the transfer vectors with the PKC theta constructs described in the preceding section. The production of recombinant Baculovirus for expression of PKC theta-FLa, PKC theta-M2a and PKC theta-M1a is performed by bacmids. All Baculovirus strains are plaque-cloned and amplified in a second amplification round in attached Sf9 cells cultured at 27° C. in 25 cm²-TC-flasks with 10 ml medium TC100 (Invitrogen)+10% FCS (Invitrogen). In a third amplification round in suspended Sf9 cells are cultured at 27° C., shaken at 90 rpm in 500 ml-Erlenmeyer flasks with 100 ml medium TC100+10% FCS+0.1% Pluronic F-68 (Invitrogen), resulting in recombinant Baculovirus suspensions with titers in the range of 0.3-2.0×10⁸ pfu/ml, determined by plaque assay.

Shake flask cultures testing the influence of various on PKC theta-expression are carried out. Suspended Sf21 cells are grown as first preculture at 27° C. and 90 rpm for three days in 100 ml medium SF900 II (Invitrogen)+1% FCS in 500 ml-Erlenmeyer flasks to reach a cell density of ca. 3×10⁶ cells/ml. For the second preculture, 90 ml of ExCell400(JRH )+1% FCS, +0.1 Pluronic F-68 medium in 500 ml-Erlenmeyer flasks are inoculated with 10 ml of the first preculture and cells are cultured for three days under the same conditions. Main cultures with a final volume of 50 ml culture with ExCell400+1% FCS, +0.1 Pluronic F-68 medium in 200 ml-Erlenmeyer flasks are inoculated with 5 ml of the second preculture and grown for ca. 3 days at 27° C., shaken at 90 rpm. When a cell density of 1.5×10⁶ cells/ml is reached, rec. Baculovirus suspension of the third amplification giving a MOI=1 and typically 5 ml of a 10× concentrated inhibitor solution is added. The cultures are then further incubated for 3 days post infection under the same conditions. At harvest, 1 ml-samples for SDS-PAGE analysis and 30 ml-samples for cell fresh weight determination are taken and centrifuged at 300 g at RT for 10 min. Cell pellets from the 1 ml-samples are taken up in 1 ml 1×SDS-PAGE buffer and an amount of such total lysates normalized for cell yield (0.2 mg fresh weight cells) is applied on the gels for SDS-PAGE analysis.

An inhibitor of PKC theta such as 3-(8-Dimethylaminomethyl-6,7,8,9-tetrahydro-pyrido-[1,2-a]indol-10-yl)-4-(1-methyl-1H-indol-3-yl)-pyrrole-2,5-dione is freshly dissolved at 4 mg/l in a solution of DMSO or 83.5% H2O+16.5% PEG 400+165 mM HCl at 60° C. in an ultrasonic cleaner to break up small particles. The resultant solution is diluted to 300 microM in culture medium and sterilized by filtration.

PKC theta-expression for production is performed either in 1 l-shake flask cultures, in a 5 l-ST-bioreactor or a 10 l-Wave-bioreactor. Precultures for all three types of production cultures are as above for 50 ml cultures. 1 l-shake flask cultures in 5 l-Erlenmeyer flasks are started by inoculating 900 ml of ExCell400+1% FCS, +0.1 Pluronic F-68 medium with 100 ml of a 3 days old second preculture and cultures are run for ca. 3 days at 28° C. and 90 rpm. 5 l-ST-bioreactor cultures in a NBS Celligen bioreactor are started by inoculating 5 l of ExCell400+1% FCS, +0.1 Pluronic F-68 medium with 300 ml of a 3 days old second preculture and bioreactor cultures are run for ca. 3 days at 28° C., agitation at 90 rpm, 1 vvm aeration with an air-oxygen gas mixture. At a density of 1.5×10⁶ cells/ml rec. Baculovirus suspension of the third amplification giving a MOI=1 and when appropriate a 10× concentrated inhibitor solution is added and cultures are continued for 72 hours under the conditions given above. PKC theta-expressing cells are harvested by two subsequent centrifugations at 400 g at RT for 20 min. first in 1 l-centrifuge bottles and then after having taken up the pelleted cells in 20 ml PBS buffer, pH 6.2 with a protease inhibitor mix (Complete, Roche) in 50 ml-plastic tubes, they are frozen and stored at −80° C. prior to extraction.

Under standard culture conditions, without the addition of an inhibitor to the culture, the majority of PKC theta versions covering only the catalytic domain, such as PKC theta-M1c are not expressed at all in rec. Baculovirus-infected insect cultures. Two ways have been found to overcome lack of PKC theta-expression for these constructs which are advantageous for structural studies. The first method is most efficient for good PKC theta-expression. It involves the addition of a specific PKC theta-inhibitor, to be added to the insect cell culture at the moment of infection with the recombinant Baculovirus preferentially at a final concentration of 30 microM. In the second method, giving only poor PKC theta-expression, the PKC theta-version covering only the catalytic domain is co-expressed with the pseudosubstrate domain (PS here referred as PKC theta-M4a).

Full length PKC theta-versions covering the catalytic domain and the regulatory domain (with the pseudosubstrate domain), such as PKC theta-FLa, are also moderately expressed in the absence of an inhibitor but are strongly expressed in the presence of an inhibitor such as 3-(8-Dimethylaminomethyl-6,7,8,9-tetrahydro-pyrido-[1,2-a]indol-10-yl)-4-(1-methyl-1H-indol-3-yl)-pyrrole-2,5-dione

The method to express PKC theta in the presence of an inhibitor is valid for different PKC theta constructs and for a broad range of the important culture conditions (inhibitor type and dose, expression time, host strain, culture medium, MOI, culture modus, culture scale).

The method may be scaled up as desired to production scale with 1-10 L-cultures. For example, 24 independent cultures at the L-scale, including 1 L-shake flasks, 5 L-ST-bioreactors or 10 L-Wave-bioreactors cultures, are used for the production of PKC theta-M1c and various other PKC versions in presence of an inhibitor. In all these cases, PKC theta expression is strong.

Gel-densitometric quantification of the expressed PKC theta-M1c (based on band intensities on Western blots with regard to PKC theta-M1a reference protein) shows that 50-120 mg/L PKC theta-M1c are produced in these cultures. The majority of the expressed PKC theta-M1c is soluble.

The SDS-PAGE analysis of cellular extracts from such PKC theta-M1c-expressing cultures shows that the majority of the expressed PKC theta-protein material has the expected molecular weight and gives a strong response on Western blots to the anti-PKC theta-antibody directed to an epitope at the C-terminus of PKC theta. The predominantly expressed PKC theta-M1c protein has a mass of ca. 42 kDa and gives a strong band on a Coomassie-stained gel co-migrating with a strong band on an anti-PKC theta-antibody treated Western blot.

### Example 5

### Purification of the Construct M1c

Due to the sensitivity of the protein all the purification steps should be on ice or at 4 C. A 10 g wet cell pellet is resuspended in app. 100 ml lysis buffer (500 mM NaCl, 50 mM Tris HCl, 5 mM Tris-(carboxyethyl) phosphine=TCEP, 1 mM NaF, 10 μl Na3VO4 pH 8.0 ), containing 4 tablets of protease inhibitor cocktail Complete Mini, EDTA free (Roche) and 100 μl Phosphatase Inhibitor cocktail 1 and 2 (Sigma). The suspension is treated with a hand homogenizer 10× and the insoluble part centrifuged 60 min with 30000 g at 4 C. The supernatant is passed through a 5 μm filter.

The supernatant is loaded on a Ni-NTA-Superflow column (Parmacia; 1 cm diameter, 3 ml volume, flow 1-3ml/min, equilibrated with lysis buffer), washed with lysis buffer plus 10 mM imidazole until absorption is close to zero (app. 15 column volumes) and eluted with a gradient from 10 to 500 mM imidazole in lysis buffer. Inhibitor containing fractions (colored) are pooled.

A stock solution of 1M MgCl2 and ATP as a powder dissolved in 1 ml water are added immediately, both to a final concentration of 10 mM. The mixture is incubated at least 18 h at 4-8 C, slightly shaking.

The phosphorylation mixture is concentrated in a precooled Ultrafree Cell MW cutoff 30000 to 1-2ml and loaded on a Superdex75 (16/60) (Pharmacia), equilibrated in SPX buffer (200 mM NaCl, 50 mM imidazole, 1 mM NaF, 5 mM TCEP, pH=8.0). Colored fractions are concentrated in an Ultrafree cell to a final protein concentration of 10-15 mg/ml (determined by HPLC).

Mass spectroscopic analysis showed that the protein preparation is sufficiently pure for crystallization after the phosphorylation. The expected mass of 41858 Dalton for the 546 residues plus the His-tag and the two phosphorylation sites is in agreement with the measurements. The calculated isoelectric point (pI) for this construct is about 6.5.

### Example 6

### Complex of PKC θ-Catalytic Domain with Inhibitor

By using an inhibitor such as 3-(8-Dimethylaminomethyl-6,7,8,9-tetrahydro-pyrido-[1,2-a]indol-10-yl)-4-(1-methyl-1H-indol-3-yl)-pyrrole-2,5-dione, the resulting protein-inhibitor complex facilitates the monitoring of the purification process. Initial purification of the wild type PKC θ (PKC theta360-706/M1a) is accomplished by three-step chromatography. Typical yields after each step are the following: ˜38 mg/L after affinity chromatography (Ni-NTA), ˜16 mg/L after size exclusion chromatography (SPX75), and ˜2 mg/L after anion-exchange chromatography (Resource-Q). MS analysis of the final purified protein reveals still a heterogeneous mixture of mono-, di -and tri-phosphorylated PKC θ.

Analysis of the total PKCθ-M1a expressed in the Baculovirus system shows that more than 80% of protein is non- or mono-phosphorylated protein. This suggests that the most soluble protein is either di- or tri-phosphorylated, whereas non-phosphorylated and mono-phosphorylated protein is unstable and precipitated upon storage, thus explaining the heavy loss of material during purification. These observations can be confirmed by dephosphorylation experiments performed on the purified PKC θ by protein phosphatase 1.

To overcome the low level of phosphorylated protein, the first phosphorylation site located in the activation loop (Thr 538) can be mutated to Glu, resulting in a constitutively active enzyme as reported previously (Newton 2001). The two auto-phosphorylation sites located in the C-terminal domain (Ser 676 and Ser 695) can be left unchanged. The expression and purification of this mutant (PKC theta360-706/I381E/T538;M1c) in presence of the inhibitor results in a mixture of non-, mono- and diphosphorylated protein. However, the overall yields of fully-phosphorylated protein recovered after anion-exchange chromatography remain low. They are less than 2-fold higher than those obtained with the wild type (M1a). The low amounts of protein (1-2 mg) obtained by this approach are not sufficient to set-up correct crystallization trials. It should be pointed out that the wild type, non-phosphorylated protein precipitates. The precipitation is temperature dependent, occurring within a few hours at room temperature or within a few days at 4° C. Further mutations of the other phosphorylation sites do not result in higher yields of homogenous protein. Purification of the triple phosphorylation-site mutant (PKC theta360-706/T538, S676E, S695E/M1g) also yields low amounts of stable protein not suitable for crystallization.

An optimized purification protocol including an auto-phosphorylation step of PKC theta360-706/M1c and the replacement of β-ME or DTT with TCEP, a much more potent reducing agent, contributes to increased stability. The yield of soluble protein can be thus increased by a factor of 50 to 1.5 mg/g wet cell paste. Once sufficient protein is available extensive screening can be performed. While the needles like crystals achieve reasonable diffraction, the hexagonal crystals show better diffraction. All crystals should be pre-frozen in the cold room at 4 C prior to measuring them at the Synchrotron.

### Example 7

### Crystallization of the Construct M1c

Two crystallization conditions can be used:

a.) Thin Needles:

Crystallization setup: Vapor diffusion, hanging or sitting drop, drop=1 ml reservoir and 1 ml protein solution. Reservoir buffer: 70 mM Tris-HCl pH=8.5, 210 mM LiSO4, 23% PEG4000. Protein solution: 10 mg/ml in SP× buffer. Crystals typically appear within 2-3 days at 4 C.

b.) Hexagonal Form:

Crystallization setup: Vapor diffusion, hanging or sifting drop, drop=1 ml reservoir and 1 ml protein solution. Reservoir buffer: 100 mM Sodium cacodylate pH=6.5, 24% MPD, 4% PEG8000. Protein solution: 10 mg/ml in SPX buffer. Crystals typically appear within 2-3 days at 4 C. Small organic molecules like 2,5-hexanediol and sulfobetaine-195 can be used as additives.

Hexagonal crystals grown at 4 C are frozen at 4 C directly by dipping them in liquid nitrogen and then transferred to the synchrotron for data collection. If the crystals are warmed up to room temperature prior to freezing they deteriorated rapidly. Images are collected with 1.0° oscillation each and a crystal-to-detector distance of 190 mm. Diffraction intensities are integrated with a MAR CCD detector on the X06SA beam line at the Swiss Light Source (wavelength=0.918396 Å). Raw diffraction data are processed using the HKL2000 software package (HKL Research Inc., Charlottesville, Va., USA) Structure factor amplitudes for negative and very weak intensities are estimated using Bayesian statistics as implemented in TRUNCATE (French and Wilson, 1978) of the CCP4 program suite (Collaborative Computational Project, Number 4, 1994).

### Example 8

### Crystal Structure of PKC Theta

The structure is determined by molecular replacement using a truncated version (residues 82-341) of the coordinates of the apo structure of the kinase domain of CAMP-dependent protein linase from Saccharomyces cerevisiae (PDB accession code 1FOT) as a search model. Molecular replacement is performed with AMoRe (Navaza 1994), using data between 9.5 and 4.0. A solution is found with 2 molecules in the asymmetric unit (correlation-coefficient 25.4%, R-factor 47.8%). For refinement and model building, the programs CNX version 2000 (Accelrys, San Diego, USA, /Brünger, 1998) and O version 7.0 (Jones et al. 1991) are used, respectively. Cross-validation is used throughout refinement. Topology and parameter files for use with CNX are generated from the idealized coordinates using the programs XPLO2D (Kleywegt and Jones, 1997). The quality of the final refined model is assessed with the programs CNX version 2000 and PROCHECK (Laskowski et al. 1993).

X-ray data were collected at the Swiss Light Source in Villigen at a wave-length of 0.918396 Å, with a crystal to detector distance of 190 mm and an exposure time of 40 sec.

Statistics on the Diffraction Data

| Synchrotron beam line | SLS (X06SA) |
|---|---|
| Temperature of data collection | 100° K |
| Number of crystals | 1 |
| Space group | P6₅ |
| Unit cell dimensions | 152.33 Å 152.33 Å 74.84 Å |
| | 90.0° 90.0° 120.00° |
| Number of monomers/asymmetric unit | 2 |
| Packing coefficient | 2.96 Å³/dalton |
| Solvent content | 56% |
| Overall B-factor from Wilson Plot | 54.42 Å² |
| (resolution range) | 3.95 Å-2.33 Å |
| Resolution range (last shell) | 50.0 Å-2.32 Å |
| | (2.36 Å-2.32 Å) |
| Number of observations | 206185 |
| Number of rejections | 2563 |
| Number of unique reflection | 42546 |
| Mosaicity | 0.263 |
| Data redundancy | 4.8 |
| Data completeness (last shell) | 99.7% (100.0%) |
| <I/σ(I)> | 10.4 |
| R_{merge}(last shell) | 6.0% (48.5%) |
| Reflections with I > 3σ(I) (last shell) | 32102 (284) |

The construct crystallized comprises residues 360 to 706 and a C-terminal tag of 6 histidine residues. There are two mutations present in this construct (I381E and T538E). The first mutation enhances the solubility and the second mutant mimics a phospho-threonine residue in order to have a constitutively active enzyme.

The folding of the molecule is into classical bilobal kinase fold. The N-terminal domain (residues 375-463) consists mainly of a five stranded β-sheet and one α-helix. The C-terminal domain (residues 464-657) consists mainly of α-helical elements. The C-terminal tail (residues 658-712) wraps around the molecule. The inhibitor is sitting between the two domains in the ATP binding site. There are many contacts between the inhibitor and the protein.

There are two protein molecules in the asymmetric unit of the crystal. The N-terminal residues 360 to 374 are disordered in both molecules. In molecule A the C-terminal tail is passing the inhibitor binding site, by making a direct contact between the side chain of tyrosine 664 and the inhibitor. In molecule B the contact area where the C-terminal tail and inhibitor could be in contact is blocked by a crystal contact to a neighboring molecule. Therefore the residues B658-B673 are displaced. No clear electron density can be found in this area, indicating that this part is disordered. The C-terminal residues A701-A712 and B712-B712 are disordered as well. The C-terminal His-tag in molecule B is partially visible, since it is stabilized by crystal contacts. A total of 648 residues have been found out of 706.

The refinement statistics indicate that the structure is refined to a reasonable R-factor (19.7%) and that the geometry is in a range as one can expect for the given resolution. The the X-ray data reveals that Cys540 is modified in both subunits. There is space for an extention of one non-hydrogen atom in the calculated electron density. Further, one MPD molecule can be identified at the interface between two neighbouring molecules. The MPD molecule has Van der Waals contacts at: A:F597, B:M467 and B:I470. The chirality of the MPD molecule cannot be determined based on the electron density at 2.32 Å resolution.

Refinement Statistics

| Data used in refinement: | |
|---|---|
| resolution range (last shell) | 20.0-2.32 Å (2.47-2.32 Å) |
| intensity cutoff (Sigma(F)) | none |
| number of reflections | 42831 |
| completeness [working + test set] (last shell) | 99.8% (99.9%) |
| test set | 5.0% |
| Fit to data used in refinement: | |
| overall R_{cryst} (last shell) | 19.7% (27.1%) |
| overall R_{free} (last shell) | 23.9% (32.4%) |
| overall mean FOM | 0.817 |
| Number of non-hydrogen atoms | |
| used in the refinement: | |
| protein atoms | 5399 |
| inhibitor atoms | 68 |
| water atoms | 413 |
| MPD | 8 |
| Overall mean temperature factor (B): | 52.6 Å² |
| (4.0 Å-2.28 Å) | |
| Overall mean temperature factor from | 43.8 Å² |
| Wilson plot | |
| RMS deviations from ideal geometry: | |
| bond length | 0.008 Å |
| bond angles | 1.0° |
| dihedral angles | 19.7° |
| improper angles | 0.69° |
| estimated standard deviation from Luzzatti | 0.27 Å (0.34 Å) |
| plot (cross validated) | |
| estimated standard deviation from sigma a | 0.28 Å (0.36 Å) |
| (cross validated) | |
| Ramachandran plot outliers | 2 |
| G factor from PROCHECK | 0.38 |

Finding optimal conditions for crystallization is difficult due to the high tendency of the protein to aggregate within hours. Once the protein is phosphorylated there is sufficient time for crystallization. The major problem is the poor solubility of the unphosphorylated protein. However, phoshorylation is just one aspect towards the improved stability. The protein is also sensitive to temperature changes. It is stable at 4° C. but crystals which are transferred to room temperature prior to freezing with liquid nitrogen are deteriorating rapidly (within about one hour).

The C-terminal tail has two different conformations which can be seen in the crystal and indicates that this loop is at least partially flexible adapting easily to the environment. The modification of Cys540 (found in both molecules in the asymmetric unit) may play a role in preventing the formation of undesired disulphide bridges. In the preparation of the protein the choice of reducing agent is important for preventing dimer formation. Prior to crystallization there was no modification of the protein as shown by mass spectroscopy. This means that it happened during crystallization. This is most likely caused by the presence of the cacodylate buffer in the reservoir solution of the crystallization screen. The kinase domain adopts an open conformation similar to that seen in PKA of Saccharomyces cerevisiae without the peptide bound.

In order to adopt a closed, liganded conformation, the domains have to rotate by about 15 degrees towards each other. The unliganded, phosphorylated PKB structure (PDB:1GZK; Yang et al. 2002) is even more open than the inhibited PKC theta structure.

The inhibitor is bound roughly as suggested by molecular modeling. The chirality of the bound enantiomer of the inhibitor, 3-(8-Dimethylaminomethyl-6,7,8,9-tetrahydro-pyrido-[1,2-a]indol-10-yl)-4-(1-methyl-1H-indol-3-yl)-pyrrole-2,5-dione is R. The three major hydrogen bonds between the succinimide moiety of the inhibitor and the protein can be confirmed. There is a water mediated hydrogen bond from the inhibitor:N15 via Wat28 to Asp508:O.

This is a feature which is impossible to predict by modeling alone. The polar group at this position is important for the solubility of the compound. Additionally, the C-terminal tail comes into close proximity of the ATP binding site. A Van der Waal's contact is formed between the side chain of phenylalanine 664 and the methylated indole fragment of the inhibitor.

The activation loop is in an activated conformation with Glu538 pointing towards solvent as one would expect for a phosphoserine residue. This means that the substrate binding groove is open and accessible to the substrate.

The electron density of the glycine rich loop is well defined. In molecule A, the two lysine residues K388 and K393 are sitting in this loop reaching towards the phosphoserine pS676. The hydrogen bonds are in the range of 3.5-3.6 Å. In molecule B, the phosphoserine residue pS676 is displaced and pointing towards solvent.

The turn motif in the C-terminal tail is phosphorylated (Ser 676) in both molecules. This phosphorylation site is reported to be crucial for the activity (Newton, 2001). In molecule B the residues 658 to 673 are not defined at all. They are displaced by a crystal contact to a neighbouring molecule and are disordered. The residues 674 to 687 are displaced relative to molecule A and have substantially higher B-factors, indicating higher mobility. In the unphosphorylated PKB, the corresponding part of the sequence is either missing in the construct or disordered. As described by A. Newton (2001), the hydrophobic motif serves in the unphosphorylated state as a recognition sequence for the up-stream kinase (PDK-1) which is assumed to phosphorylate the activation loop. Once the hydrophobic motif is phosphorylated, it is masked and no longer able to bind PDK-1. The highly variable C-terminal residues following the hydrophobic motif are important for the subcellular distribution of the kinases (Newton, 2001). The two phenylalanine residues (691, 694) prior to the phosphorylation site (Ser 695) serve as a structural anchor to the N-terminal domain. A large portion of the tail tracing varies substantially, but the striking feature is that the hydrophobic motif is structurally preserved and it is anchored in the same position in the N-terminal lobe. This motif is not only highly conserved in the family of PKC but also in families of PKA and PKB. In the PKA structures (1ATP and 1FOT) the hydrophobic motif bound in exactly the same way as seen in PKC theta. The phosphorylation site of PKC theta is absent in PKA since the amino acid chain ends at the second phenylalanine residue of the hydrophobic motif. In the PKB structures reported by Yang et al. (2002) the non-phosphorylated hydrophobic motif is claimed to be present in the construct, but is disordered in the structure. It may be assumed that the non-phosphorylated hydrophobic motif retains its potential for interaction with the N-terminal lobe of the structure. Nevertheless electron density is not found for this part of the structure indicating that this part is flexible. In PKC, we found the hydrophobic motif bound to the N-terminal lobe in its phosphorylated form. This indicates that the assumption that the non-phosphorylated form binds to the N-terminal lobe is unlikely. It is much more likely that the phosphorylated form binds and the non-phosphorylated is flexible. This would also be a good explanation why the non-phosphorylated protein has such a high tendency to aggregate. In PKA, the hydrophobic motif bound in exactly the same way as in PKC θ. The phosphorylation site after the hydrophobic motif is missing in PKA, but the negative charge of the C-terminus may compensate for it.

In both molecules, Cys540, which is solvent accessible, is found to be either methylated or oxidated. Methylation can be caused by the cacodylate buffer originating from the reservoir solution in the crystallization screen. Mass spectroscopy done prior to the crystallization setup did not detect any modifications.

Contact distances between the inhibitor and the binding site.

| Inhibitor | Inhibitor | Protein | Distances in | Distances in |
|---|---|---|---|---|
| group | atoms | Atoms | Molecule A | Molecule B |
| 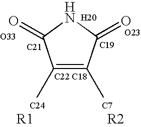 | N20 O23 O23 O23 O33 O33 O33 C21 | Glu 459:O Thr 442:OG1 Met 458:CE Wat 11:O Leu 461:N Leu 461:O Tyr 460:CD1 Ala 407:CB | 2.84 2.63 3.69 3.02 2.66 3.37 3.53 3.77 | 2.80 2.54 3.36 3.21 2.63 3.34 3.26 3.68 |
| 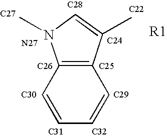 | C27 C27 C27 C32 C32 C31 | Phe 664:CG Wat 10:O Wat 13:O Val 394:CB Phe 391:CZ Gly 387:CA | 3.34 3.43 3.48 3.78 3.60 3.34 | —3.49 3.50 3.82 3.76 3.53 |
| 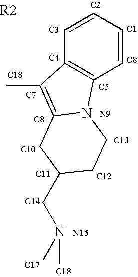 | N15 N15 C10 | Wat 28:O Asp 508:O Leu 511:CD1 | 2.75 3.52 3.89 | 2.80 3.41 3.98 |

### REFERENCES

### References Related to PKC

Altman A, Isakov N, Baier G. Protein kinase C theta: a new essential superstar on the T-cell stage. Immunology Today. 2000; 21: 567-573.
Arendt C W, Albrecht B, Soos T J, Littman D R. Protein kinase C theta: signaling from the center of the T-cell synapse. Current Opinion in Immunology. 2002; 14: 323-330.
Baier, G. Telford, D. Giampa, L., Coggeshall, K. M., Baier-Bitterlich, G., Isakov, N. Altman, A. (1993) Molecular cloning and characterisation of PKC theta, a novel member of the protein kinase C (PKC) gene family expressed predominantly in hematopoietic cells” J. Biol. Chem. 268(7), 4997-5004.
Chang, J D, Xu, Y, Raychowdhury, M K, Ware, J A (1993) Molecular cloning and expression of the cDNA encloding a novel isoenzyme of protein kinase C (nPKC). A new member of the nPKC family expressed in skeletal muschle, megakaryoblastic cells and platelets. J. Biol. Chem. 268, 14208-14214.
Chang, J D, Xu, Y, Raychowdhury, M K, Ware, J A (1994) Correction, J. Biol. Chem. 269, 31322-31322.
Coudronniere N, Villalba M, Englund N, Altman A. NF-kB activation induced by T cell receptor/CD28 costimulation is mediated by protein kinase C-theta. PNAS. 2000; 97: 3394-3399.
Dienz O, Hehner S P, Dröge W, Schmitz M L. Synergistic activation of NF-kB by functional cooperation between Vav and PKC theta in T lymphocytes. J. Biol. Chem. 2000; 275: 24547-24551.
Dekker L V, Palmer R H, Parker P J. The protein kinase C and protein kinase C related gene families. Curr. Opin. Struct. Biol. 1995; 5: 396-402
Isakov N & Altman A. (2000). “Protein kinase C in T cell activation.” Annual Review of Immunology 20, 761-794.
Khoshnan A, Bae D, Tindell C A, Nel A E. The physical association of protein kinase C theta with a lipid raft-associated inhibitor of kB factor kinase (IKK) complex plays a role in the activation of the NF-kB cascade by TCR and CD28. J. Immunology. 2000; 165: 6933-6940.
Lin X, O'Mahony A, Mu Y, Geleziunas R, Greene W C. Protein kinse C-theta participates in NF-kB activation induced by CD3-CD28 costimulation through selective activation of IkB kinase beta. Mol. Cell. Biol. 2000; 20: 2933-2940.
Meller N, Altman A, Isakov N. New prespectives on PKC theta, a member of the novel subfamily of protein kinase C. Stem Cells. 1998; 16: 178-192.
Monks C R F, Kupfer H, Tamir I, Barlow A, Kupfer A. Selective modulation of protein kinase C-theta during T-cell activation. Nature. 1997; 385: 83-86.
Monks C R F, Freiberg B A, Kupfer H, Sciaky N, Kupfer A. Three-dimensional segregation of supramolecular activation clusters in T cells. Nature. 1998; 395: 82-86.
Newton A C (2001). Protein kinase C: structural and spatial regulation by phosphorylation, cofactors, and macromolecular interactions. Chem. Rev., 101(8):2353-2364.
O'Reilly, D. R., Miller, L. K. and Luckow, V. A. (1994) Baculovirus Expression Vectors, Oxford University Press, New York, N.Y.
Pappa H, Murray-Rust J, Decker L V, Parker P J & McDonald N Q (1998). “Crystal structure of the C2 domain from protein kinase C-theta.” Structure, 6, 885-894.
Sun Z, Arendt C W, Ellmeier W, Schaeffer E M, Sunshine M J, Gandhi L, Annes J, Petrzilka D, Kupfer A, Schwartzberg P L, Littman D R. PKC-theta is required for TCR-induced NF-kB activation in mature but not immature T lymphocytes. Nature. 2000; 404: 402-407.
Sutton R B & Sprang S R (1998). “Structure of the protein kinase C theta phospholipid-binding C2 domain complexed with Ca²⁺.” Structure 6, 1395-1405.
Verdaguer N, Corbalan-Garcia S, Ochoa W F, Fita I & Gómez-Fernández J C (1999). “Ca²⁺ bridges the C2 membrane-binding domain of protein kinase C theta directly to phosphatidylserine.” EMBO J., 18, 6329-6338.
Wilkinson S E, Nixon J S. T-cell signal transduction and the role of protein kinase C. Cell. Mol. Life Sci. 1998; 54: 1122-1144.
Williams D H & Mitchell T (2002). “Latest developments in crystallography and structure-based design of protein kinase inhibitors as drug candidates.” Current Opinion in Pharmacology 2:1-7.
Yang J, Cron P., Thompson V, Good V M, Hess D, Hemmings B A & Barford D (2002). “Molecular Mechanism for the Regulation of Protein Kinase B/Akt by hydrophobic Motif Phosphorylation”. Molecular Cell 9, 1227-1240.
Zhang G, Kazanietz M G, Blumberg P M & Hurley J H (1995). “Crystal structure of the Cys2 Activator-Binding domain of protein Kinase C theta in complex with phorbol ester.” Cell 81, 917-924.

### REFERENCES

Related to Computational Programs and Methods:

Anand K, Pal D & Hilgenfeld R (2002). ,,An Overview on 2-methyl-2,4-pentanediol in crystallization and in crystals of biological macromolecules.” Acta Cryst. D58, 1722-1728.
Ausubel F. M., Brent R., Kingston R. E., Moore D. D., Seidmann J. G., Struhl K. “Current protocols in molecular biology.” Revised edition. New York: John Wiley & Sons, 1997.
Brünger A. T. (1996). XPLOR version 3.843, Yale University, New Haven, Conn.
Collaborative Computational Project, Number 4 (1994). “The CCP4 suite: Programs for Protein Crystallography.” Acta Cryst, D50, 760-763.
Geiser M, Cèbe R, Drewello D & Schmitz R (2001). “Integration of PCR Fragments at Any Specific Site within Cloning Vectors without the Use of Restriction Enxymes and DNA Ligase.” Biotechniques 31, 88-92.
Jones, T. A., Zou, J. Y., Cowan, S. W. & Kjeldgaard M (1991). “Improved methods for binding protein models in electron density maps and the location of errors in these models.” Acta Cryst., A47, 110-119.
French G. S., Wilson, K. S. (1978). “On the Treatment of Negative Intensity Observations.” Acta Cryst A34: 517-525.
Kleywegt G. J. & Jones T. A. (1997). Model Building and Refinement Practice. Methods Enzymology 277: 208-230.
Laskowski, R. A., MacArthur M. W., Moss, D. S. & Thornton, J. M. (1993). “PROCHECK: a program to check the stereo chemical quality of protein structures.” J. Appl. Crystallogr., 26, (2) 283-291.
Navaza, J. (1994). “AMoRe: an automated package for molecular replacement.” Acta Cryst. A50, 157-163.
Otwinowski Z. & Minor W. (1997) Methods in Enzymology, Volume 276: Macromolecular Crystallography, part A, p. 307-326, C. W. Carter, Jr. & R. M. Sweet, Eds., Academic Press. “Processing of X-ray Diffraction Data Collected in Oscillation Mode.”

1. A crystal of the PKC theta comprising the catalytic domain of PKC theta with a unit cell dimension of a=152.33±5 Ångstroms, b=152.33±5 Ångstroms, c=78.84±5 Ångstroms; α90.0 degrees, β=90.0 degrees, γ=120.0 degrees.

2. A crystal of the PKC theta comprising the catalytic domain of PKC theta wherein said catalytic domain has a three-dimensional structure comprising the atomic structure coordinates of Table 1.

3. A crystal of claim 1 wherein the catalytic domain of PKC theta comprises the sequence of SEQ ID. No. 2, a fragment or a homologue thereof.

4. A crystal of a mutant of the catalytic domain of PKC theta wherein said mutant is selected from the group of mutants SEQ ID No. 3 to SEQ ID No. 20, a fragment or homologue thereof.

5. A crystal of claim 3 wherein the catalytic domain of PKC theta comprises at least the ATP-binding site.

6. A crystal of claim 1 bound to at least one ligand or low molecular weight compound.

7. A computer readable medium comprising data storage material encoded with computer readable data wherein said data comprises the atomic coordinates of Table 1 comprising the catalytic domain of PKC theta.

8. A method for making a crystal of a PKC theta comprising the steps of:
(i) purification of the full-length PKC theta of SEQ ID No.1
(ii) expression of the full-length PKC theta (SEQ ID No.1) or expression of the catalytic domain of PKC theta (SEQ ID No.2) in a suitable host cell
(iii) purification of the desired PKC theta domain.

9. A method of making a crystal of PKC theta according to claim 8 wherein in step (ii) expression occurs in the presence of a PKC theta inhibitor.

10. A method of making a crystal according to claim 8 wherein the catalytic domain of PKC theta is phosphorylated at sites Serine 676 or Serine 695 or both sites.

11. A method of making a crystal according to claim 8 wherein the catalytic domain of PKC theta (SEQ ID No. 2), a mutant (SEQ ID No. 3 to SEQ ID No. 20), fragment, homologue of the catalytic domain of PKC theta or an N-terminal extended catalytic domain of PKC theta is used.

12. A method according to claim 8 wherein the catalytic domain of PKC-theta (SEQ ID No. 2), a mutant (SEQ ID No. 3 to SEQ ID No. 20), fragment, homologue of the catalytic domain of PKC-theta or N-terminal extended catalytic domain of PKC theta is bound to at least one ligand or low molecule weight chemical compound at any step prior to crystallisation.

13. A method of determining the three-dimensional structure of the catalytic domain of PKC theta comprising:
(i) crystallisation of PKC theta comprising the catalytic domain of PKC theta (SEQ ID No.2), a mutant of the catalytic domain of PKC theta (SEQ ID No.3 to SEQ ID No. 20), fragment, homologue of the catalytic domain of PKC theta or N-terminal extended catalytic domain of PKC theta is used
(ii) utilizing the atomic coordinates of Table 1 in whole or in part to determine the three-dimensional structure of the catalytic domain of PKC theta.

14. A method for determining the three-dimensional structure of a complex comprising the catalytic domain of PKC theta (SEQ ID No.2), a mutant (SEQ ID No. 3 to 20), fragment, homologue of the catalytic domain of PKC theta or N-terminal extended catalytic domain of PKC theta is bound to at least one ligand comprising the steps of:
(i) obtaining x-ray diffraction data for crystals of the complex
(ii) utilizing the atomic coordinates of Table 1 in whole or in part to define the three-dimensional structure of the complex.

15. A method of identifying a ligand or low molecular weight compound that binds to the catalytic domain of PKC theta comprising:
(i) using the three dimensional structure of the catalytic domain of PKC theta derived in whole or in part from the set of atomic coordinates in Table 1
(ii) selecting a ligand or low molecular weight compound that binds to the catalytic domain of PKC theta.

16. A method of identifying a ligand or low molecular weight compound that binds to the catalytic domain of PKC theta according to claim 15 wherein the catalytic domain of PKC theta comprises at least the ATP-binding site of said domain.

17. A method of claim 15 for use in selecting ligands which inhibit the activity of PKC theta.

18. A method of designing a ligand or low molecular weight compound capable of binding to PKC theta catalytic domain comprising:
(i) using the atomic coordinates of Table 1 in whole or in part to determine the three dimensional structure of PKC theta catalytic domain
(ii) probing said catalytic domain of PKC theta with candidate ligands or low molecular weight compounds to determine which bind to the catalytic domain of PKC theta
(iii) selecting those ligands or low molecular weight compounds which bind to the catalytic domain of PKC theta
(iv) modifying those ligands or low molecular weight compounds which bind to maximize physical properties such as solubility, affinity, specificity or potency.

19. A method of designing a ligand according to claim 18 wherein the candidate ligands or low molecular weight compounds are screened in silico.

20. A method of designing a ligand or low molecular weight compound capable of binding to a PKC family member using the atomic coordinates of Table 1 in whole or in part to determine the three dimensional structure of a PKC family member's catalytic domain.

21. A method according to claim 18 for use in designing ligands which inhibit the activity of PKC theta.

22. A pharmaceutical composition comprising a ligand identified or designed by the methods of claim 15 for use in preventing or treating of diseases and conditions involving T-lymphocytes and/or a PKC family member.
